(19) United States
(12) Patent Application Publication
(10) Pub. No.: US 2002/0110827 A1
(43) Pub. Date: Aug. 15, 2002

(54) Quantitative mRNA amplification

(76) Inventors: Craig P. Hunter, Arlington, MA (US); Larry Ryan Baugh, Somerville, MA (US)

Correspondence Address: HALE AND DORR, LLP, 60 STATE STREET, BOSTON, MA, 02109

(21) Appl. No.: 10/038,177

(22) Filed: Dec. 21, 2001

Related U.S. Application Data
(60) Provisional application No. 60/268,102, filed on Feb. 12, 2001.

(51) Int. Cl. C12Q 1/68; C12P 19/34

(52) U.S. Cl. 435/6; 435/91.2

(57) ABSTRACT

The present invention relates to methods for copying and amplifying complex populations of mRNA molecules. Specifically, the invention relates to methods for copying and amplifying complex populations of mRNA molecules that maximize the retention of representative information in populations of copied mRNA molecules.

### RELATED APPLICATIONS

This application claims the benefit of U.S. Provisional Application Serial No. 60/268,102, filed Feb. 12, 2001, the entire contents of which are incorporated herein.

### FIELD OF THE INVENTION

This invention relates to molecular biology methods for synthesizing cDNA and amplifying mRNA. This invention also relates to methods for enhancing the in vitro processivity of reverse transcriptase.

### BACKGROUND OF THE INVENTION

Important biological insights can be gained by examining the identities and levels of expression of genes expressed by a particular cell or population of cells. The presence, absence, or abundance of certain transcripts may reflect a particular differentiated state, stage in the cell cycle, transformation to a neoplastic state, viral infection, or other condition. Thus, methods that probe gene expression in living cells are useful not only for providing an understanding of the fundamental processes of living thing, but can also provide molecular methods for diagnosing disease by detecting transcripts associated with cancers, infectious diseases, or other conditions. Conventional methods for detecting, characterizing, and quantifying expressed transcripts include northern blotting, S1 nuclease analysis, RT-PCR, primer extension, and RNase protection.

The proliferation of genomic sequence information has driven the development of new technology for comprehensive examination of all the RNA transcripts expressed by living cells, providing yet another method for probing gene expression. This new methodology, known as transcript profiling, permits the identification of functional groups of genes by comparing the expression profiles of different organisms, cell types, or cells taken at different stages in the cell cycle or the life cycle of an organism. DNA microarrays provide a powerful tool for probing gene expression in organisms for which the genome sequence is available (see e.g., Lockhart and Winzeler (2000) Nature 405: 827-836; Young (2000) Cell 102: 9-15).

However, many challenges stand in the way of obtaining adequate amounts of material for analysis of gene expression in multicellular organisms. In order to examine the unique expression patterns in different cells and tissues within a multicellular organism, a means of isolating or enriching mRNA from the cell types of interest is essential. Methods for dissection and microdissection, developmental staging, and cell sorting provide some means of obtaining enriched samples of particular cell types. However, obtaining sufficient quantities of mRNA for analysis of gene expression from cells isolated using these methods may not be feasible, especially for cells that are rare or difficult to isolate, such as particular neurons, small tumor samples, or cells from early stages of development. Both conventional methods, such as northern hybridization and S1 nuclease analysis, and more recently developed transcript profiling methods require microgram amounts of total RNA to provide accurate results. To pursue a better understanding of gene expression in higher organisms, a technique capable of amplifying small amounts of mRNA while preserving the information contained in the sample is needed.

Existing, widely used amplification strategies may fail to faithfully preserve the information content of mRNA harvested from living cells. Analyses that probe the relative amounts of transcripts in a given cell or cell type require that any amplification strategy not result in the over-representation of some transcripts and the under-representation of others in the amplified product. Rare transcripts and those that are not readily amplified should be preserved and their representation in the amplified population should reflect their levels of expression in the organism. Similarly, abundant transcripts and those that are easily amplified should not be over-represented in the amplified product. Any biases that might alter the representation of certain species in the sample should therefore be reduced as much as possible.

For the aforementioned reasons, the polymerase chain reaction is not readily applicable to amplification of small amounts of material for assessing the relative levels of expression of different genes. While the substantial increase in material achieved by the polymerase chain reaction is attractive, the geometric kinetics will amplify sequence-dependent and copy-number dependent biases geometrically (Peccoud and Jacob (1996) Biophysical Journal 71: 101-108), and therefore will significantly distort the information content in the process of amplification.

In contrast, the biases of an isothermal, asymmetric amplification reaction should be limited to modest sequence-dependent biases, and should permit more reliable and quantitative detection of differences between samples. mRNA amplification based on in vitro transcription of mRNA has been shown by conventional methods to amplify transcripts known to have low or moderate levels of expression (VanGelder et al. (1990) Proc. Natl. Acad. Sci. USA 87: 1663-1667). However, the ability of such a protocol to linearly amplify small amounts of mRNA while maintaining relative mRNA levels and successfully amplifying rare transcripts has not been demonstrated. In order to accurately examine gene expression in multicellular organisms, a protocol for amplifying mRNA from small amounts of starting material while preserving the information content of the sample is needed.

### SUMMARY OF THE INVENTION

A linear amplification procedure has been discovered that maximizes the preservation of the information content of a complex population of RNA molecules. The present invention exploits this discovery to provide methods of amplifying complex populations of RNA molecules while preserving the information content of the population.

Accordingly, in a first aspect, the invention provides a method for faithfully amplifying a complex population of RNA molecules which reduces the generation of template-independent amplification products. By limiting the generation of template-independent products, the information content of the population is more faithfully preserved. The RNA molecules are amplified by generating cDNA copies, from which antisense RNA (aRNA) is generated by in vitro transcription.

By “complex population of RNA molecules” is meant a collection of RNA molecules comprising non-identical species of RNA molecules. The molecules are non-identical in length, nucleotide sequence, secondary structure, and/or relative representation within the population. The population may consist of any number of species greater than one. In at least some embodiments of the invention, the RNA sample is total RNA extracted from a living cell or cells.

The term “amplification” as used herein means copying a nucleic acid molecule or a collection of nucleic acid molecules such that the total number of nucleic acid molecules is increased.

“Total RNA” as used herein means the ribonucleic acid isolated from a living cell or cells which has not been subjected to a procedure for fractionating certain RNA components.

By “total information content” is meant herein the total collection of transcripts corresponding to expressed genes. Thus, preserving total information content means maintaining the presence of both abundant and rare transcripts during the amplification protocol. In at least some embodiments, transcripts present at a frequency of 100 parts per million are preserved; in at least some other embodiments, transcripts present at a frequency of 10 parts per million are preserved; in at least some other embodiments, transcripts present at a frequency of 5 parts per million are preserved, and in at least some other embodiments, transcripts present at a frequency of 3 parts per million are preserved.

By “relative representation” is meant the relative abundance of a transcript in a sample compared to other transcripts in the sample. Thus, preserving relative representation means maintaining the abundance of the transcripts in a population during a copying or amplification protocol. Thus, in at least some embodiments, the correlation coefficient between an amplified sample and its unamplified counterpart is at least 0.90, in at least some other embodiments, the correlation coefficient between an amplified sample and its unamplified counterpart is at least 0.95, in at least some other embodiments, the correlation coefficient between an amplified sample and its unamplified counterpart is at least 0.97, and in at least some other embodiments, the correlation coefficient between an amplified sample and its unamplified counterpart is 0.99.

As used herein, “faithfully amplifying” means that the amplification of the mRNA preserves either the total information content or the relative representation of the mRNAs in the population, or preserves both the total information content and the relative representation of the population of mRNA molecules.

By “template-independent products” is meant nucleic acid species that are not copied from the original RNA template from the first step, or from the cDNA template generated by the reverse transcriptase. Template-independent products can be generated in the absence of mRNA if all other standard reaction components, such as primer, nucleotide triphosphates, buffer, reverse transcriptase, and/or RNA polymerase, for the cDNA synthesis and/or the in vitro transcription reaction are present.

In a second aspect of the present invention, the invention provides a method for amplifying a complex population of mRNA molecules, comprising generating cDNA from a complex population of RNA molecules and in vitro transcribing the cDNA to generated amplified antisense RNA. The first-strand synthesis reaction for generating cDNA comprises a primer complementary to the mRNA molecules. In this aspect of the invention, the concentration of primer used to direct first-strand synthesis in step (a) does not exceed 0.2 μM in step (b). In at least some embodiments, the primer from step (a) is present at a concentration of no greater than 0.02 μM in step (b). In at least some other embodiments, the primer is present at a concentration of no greater than 1 μM in step (a). In at least some embodiments, the mRNA molecules are amplified from no more than 10 μg of total RNA, in at least some other embodiments, the mRNA molecules are amplified from no more than 5 82 g of total RNA, in at least some other embodiments, the mRNA molecules are amplified from no more than 1 μg of total RNA, in at least some other embodiments the mRNA molecules are amplified from no more than 100 μg of total RNA, in at least some other embodiments, the mRNA molecules are amplified from no more than 10 μg of total RNA, and in at least some other embodiments, the mRNA molecules are amplified from no more than 2 μg of total RNA. In at least some embodiments, the mRNA molecules are amplified from the total RNA isolated from fewer than 1000 cells, in at least some other embodiments, the mRNA molecules are amplified from the total RNA isolated from fewer than 100 cells, in at least some other embodiments, the mRNA molecules are amplified from the total RNA isolated from fewer than 10 cells, and in at least some other embodiments, the mRNA molecules are isolated from the total RNA isolated from a single cell.

In a third aspect of the present invention, the invention provides a method for amplifying a complex population of RNA molecules which increases the average length of the amplified products. The RNA molecules are amplified by generating cDNA copies in the presence of a single-strand binding protein at a concentration sufficient to support completed synthesis by a reverse transcriptase of mRNA templates greater than 600 nucleotides in length, from which antisense RNA (aRNA) is generated by in vitro transcription. In this aspect of the invention, the primer used in first-strand cDNA synthesis is present at a concentration of no greater than 0.2 μM during in vitro transcription. In at least some embodiments, the single-strand binding protein is present at a concentration of at least 0.015 μM. In at least some other embodiments, the single-strand binding protein is present at a concentration of at least 0.0061 μM.

The phrase “single-strand binding proteins” encompasses proteins known to be associated with nucleic acid polymerization which bind to single-stranded nucleic acids with high affinity and with low sequence specificity. In at least some embodiments of the second aspect, the single-strand binding protein is T4 gp32. In at least some other embodiments of the second aspect, the single-strand binding protein is the single-strand binding protein of Escherichia coli.

In at least some embodiments of the third aspect of the invention, the primer from step (a) is present at a concentration of no greater than 0.02 μM in step (b). In at least some other embodiments, the primer is present at a concentration of no greater than 1 μM in step (a). In at least some embodiments, the mRNA molecules are amplified from no more than 10 μg of total RNA, in at least some other embodiments, the mRNA molecules are amplified from no more than 5 μg of total RNA, in at least some other embodiments, the mRNA molecules are amplified from no more than 1 μg of total RNA, in at least some other embodiments the mRNA molecules are amplified from no more than 100 ng of total RNA, in at least some other embodiments, the mRNA molecules are amplified from no more than 10 ng of total RNA and in at least some other embodiments, the mRNA molecules are amplified from no more than 2 ng of total RNA. In at least some embodiments, the mRNA molecules are amplified from the total RNA isolated from fewer than 1000 cells, in at least some other embodiments, the mRNA molecules are amplified from the total RNA isolated from fewer than 100 cells, in at least some other embodiments, the mRNA molecules are amplified from the total RNA isolated from fewer than 10 cells, and in at least some other embodiments, the mRNA molecules are isolated from the total RNA isolated from a single cell.

In a fourth aspect of the present invention, the invention provides a method for amplifying a complex population of mRNA molecules from a starting sample of no more than 100 ng of total RNA. The RNA molecules are amplified by generating cDNA copies in the presence of a single-strand binding protein at a concentration sufficient to support completed synthesis of RNA templates greater than 600 nucleotides in length, from which antisense RNA is generated by in vitro transcription. A second round of amplification is performed, in which cDNA is synthesized from the aRNA of the first round in the presence of a single-strand binding protein at a concentration sufficient to support completed synthesis of RNA templates greater than 600 nucleotides in length by reverse transcriptase, and in which the cDNA of the second round is in vitro transcribed to generate an increased amount of aRNA.

In at least some embodiments of the fourth aspect of the invention, the primer from step (a) is present at a concentration of no greater than 0.02 μM in step (b). In at least some other embodiments, the primer is present at a concentration of no greater than 1 μM in step (a). In at least some embodiments, the mRNA molecules are amplified from no more than 10 ng of total RNA and in at least some other embodiments, the mRNA molecules are amplified from no more than 2 ng of total RNA. In at least some embodiments, the mRNA molecules are amplified from the total RNA isolated from fewer than 1000 cells, in at least some other embodiments, the mRNA molecules are amplified from the total RNA isolated from fewer than 100 cells, in at least some other embodiments, the mRNA molecules are amplified from the total RNA isolated from fewer than 10 cells, and in at least some other embodiments, the mRNA molecules are isolated from the total RNA isolated from a single cell.

In at least some embodiments of the fourth aspect of the invention, the single-strand binding protein is present at a concentration of at least 0.015 mM. In at least some other embodiments, the single-strand binding protein is present at a concentration of at least 0.0061 mM. In at least some embodiments, the single-strand binding protein comprises T4 gp32. In at least some other embodiments, the single-strand binding protein comprises the single-strand binding protein of Escherichia coli.

In a fifth aspect of the present invention, the invention provides a method for increasing the average length of DNA molecules synthesized by a reverse transcriptase by including a single-strand binding protein at a high concentration in the reverse transcription reaction. In at least some embodiments, the single-strand binding protein is T4 gp32. In at least some embodiments, the single-strand binding protein is the single-strand binding protein of Escherichia coli. In at least some embodiments of the invention, the single-strand binding protein is present at a concentration of at least 0.015 mM. In at least some embodiments of the invention, the single-strand binding protein is present at a concentration of at least 0.0061 mM.

In a sixth aspect of the present invention, the invention provides kits for the synthesis of cDNA, which comprise a primer or primers for first-strand synthesis, a single-strand binding protein at high concentration, and a reverse transcriptase. In at least some embodiments, the kit comprises instructions for use. In at least some embodiments, the kit comprises instructions for limiting the synthesis of template-independent products. In some embodiments, the kit further comprises instructions for increasing the processivity of the reverse transcriptase, and control RNA molecules for the measurement of increased processivity of the RNA polymerase. In at least some embodiments, the kit comprises a single strand binding protein at a concentration of at least 0.25 mM. In at least some embodiments, the kit comprises ribonucleic acid standards for measuring the processivity of the reverse transcriptase. In at least some embodiments, the ribonucleic acid standards include molecules more than 4000 nucleotides in length.

### BRIEF DESCRIPTION OF THE DRAWING

The invention is illustrated with reference to the following drawing, which are not intended to be limiting to the invention and in which:

FIG. 1 is a photographic representation of amplified antisense RNA separated by agarose gel electrophoresis and visualized by SYBR gold staining;

FIG. 2 is a graphic representation of comparisons of gene frequencies from samples independently amplified from the same starting material hybridized to DNA microarrays;

FIG. 3 is a graphic representation of comparisons of transcript frequencies from the same starting material serially diluted, amplified, and hybridized to DNA microarrays;

FIG. 4 is a graphic representation of comparisons of gene frequencies from two different samples serially diluted, amplified, and hybridized to DNA microarrays; and

FIG. 5 is a photographic representation of cDNA synthesized with increasing amounts of a single-strand binding protein separated by agarose gel electrophoresis and visualized with SYBR gold staining.

### DETAILED DESCRIPTION OF THE INVENTION

The present invention provides methods for creating cDNA from complex populations of mRNA molecules while maintaining the information content of the population. In at least some aspects of the invention, the cDNA is used as a template for creating amplified antisense RNA (aRNA), and the information content is maintained through the amplification protocol. Such methods are useful for applications that require microgram quantities of RNA from sources that do not readily yield microgram amounts of material. Thus, the invention provides a means of generating material for a number of methods that probe the information content of complex populations of mRNA, including transcript profiling using microarrays, northern hybridization, S1 nuclease assays, RNase protection assays, RT-PCR, and the creation of cDNA libraries. The amplification methods according to the invention can also be used to selectively amplify specific transcripts from complex populations of RNA to probe the relative expression of a limited number of genes. Such methods can be used for the detection of viral, bacterial, or parasitic transcripts, or to examine the relative expression of a select group of genes.

The methods of the invention generate cDNA from complex populations of RNA molecules, for example, from total RNA harvested from living cells. The complex population of mRNA may be derived from a variety of eukaryotic sources including, but not limited to, single-celled organisms such as yeast, protozoa, or single-celled algae, or multicellular organisms, including plants and animals. RNA derived from a multicellular organism may represent a single tissue or cell type, or may comprise multiple cell types. Methods for isolating RNA from cells, tissues, organs, or whole organisms are known to those of skill in the art (see, e.g. Maniatis et al. (1989) Molecular Cloning: A Laboratory Manual 2d. Ed., Cold Spring Harbor Press, Cold Spring Harbor, N.Y.; and Ausubel et al. (1990) Current Protocols in Molecular Biology, John Wiley & Sons, New York, N.Y.).

The double-stranded cDNA is synthesized in two steps. In first-strand synthesis, the reverse transcriptase creates a DNA molecule, which is complementary to the mRNA molecule. The resulting DNA molecule serves as a template for second-strand synthesis, which creates a DNA molecule having the same nucleotide sequence as the original mRNA transcript.

First-strand synthesis is carried out by a reverse transcriptase and directed by a primer or primers that hybridize to the mRNA molecule to be copied. A variety of priming strategies can be used. Non-limiting examples of primers according to the invention include primers which comprise sequences complementary to the polyadenine sequences found at the 3′ end of mRNA molecules, and other sequence-specific primers designed to selectively amplify certain RNA species, as well as collections of random primers, such as random hexamers, which will hybridize to various locations in the targeted population. The primer may also include a promoter for an RNA polymerase, which is useful for directing RNA polymerization in later amplification step or steps.

In certain methods of the invention, the cDNA molecules are copied and amplified by an RNA polymerase to generate amplified antisense RNA (aRNA). In methods where amplified antisense RNA is produced, the double-stranded cDNA molecule is used as a template for RNA synthesis. Transcription of the second-strand of the cDNA, promoted by the transcriptional start site incorporated with the primer, generates RNA molecules which are antisense copies of the original mRNA transcript, meaning that they encode the sequence which is complementary to the original mRNA sequence (the antisense sequence) rather than the original mRNA sequence itself (the sense sequence). The complete process as described above produces up to 700-fold amplification when a single round of amplification is performed according to the methods of the invention. When a second round of amplification is added to the protocol, the complete process produces 33,000-167,000 fold amplification.

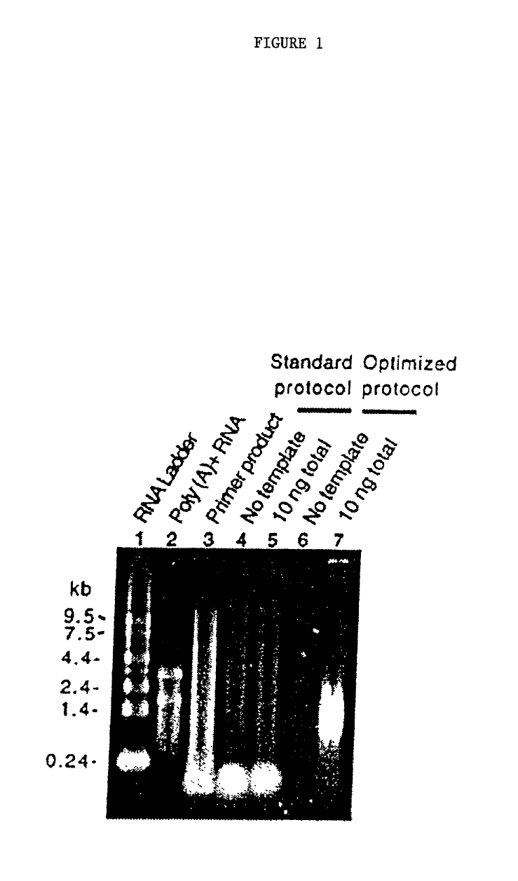

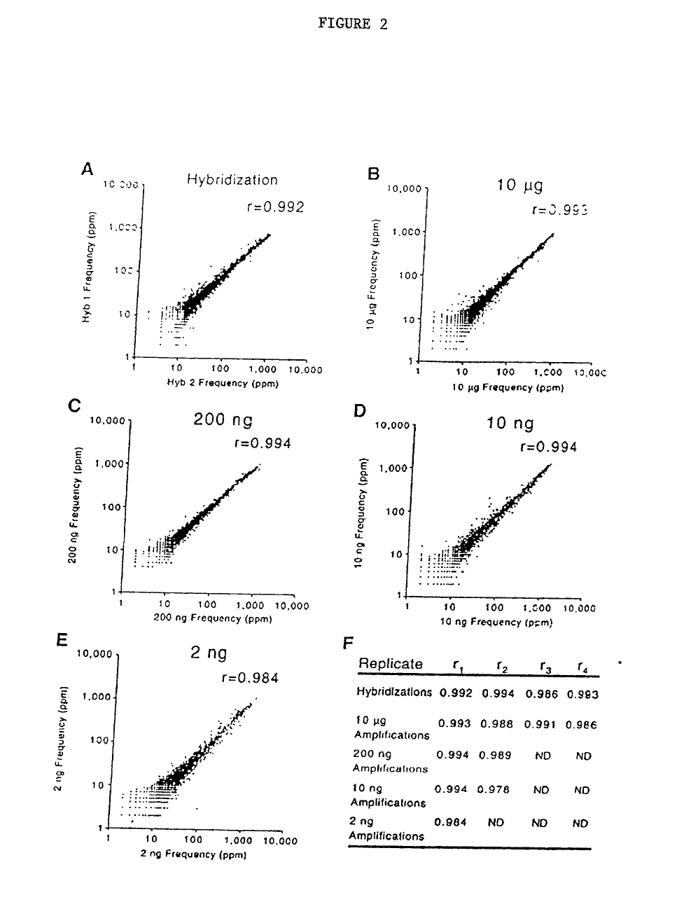

The present invention provides preservation of total information content and/or faithful RNA amplification. Standard amplification protocols such as those disclosed in Wang et al. (Nature Biotechnology (2000) 18: 457-459) and Luo et al. (Nature Medicine (1999) 5: 117-122) have been used to amplify mRNA from small amounts of starting material, but the methods of both studies fail to preserve information through the amplification protocol. This is established by a systematic examination of the standard amplification protocol disclosed by Wang et al. and Luo et al. and identification of abundant template-independent products. As shown herein in FIG. 1, the standard amplification protocol generates template-independent products, which can be visualized through agarose gel electrophoresis and SYBR gold staining. The standard protocol is compared to an optimized protocol, which reduces the template-independent product (also shown in FIG. 1) and which improves the retention of information content, as described below herein and shown in FIGS. 2-4.

Template-independent products are defined as products that are generated by the enzymes in the amplification reaction regardless of whether a sample comprising mRNA molecules, e.g., the “template,” has been added. Examination of conditions under which template-independent products were formed demonstrated that T7 RNA polymerase efficiently synthesized template-independent products in an in vitro amplification reaction when the only polynucleotide present was the primer for cDNA synthesis. Furthermore, the template-independent products were synthesized with T3 polymerase as well, suggesting that the elimination of these products could not be readily accomplished by substituting another RNA polymerase in the amplification reaction. It was also found that the contaminating product was not readily eliminated by HPLC purification of the primer, and could not be eliminated by altering the primer sequence.

It has been surprisingly discovered that the generation of these template-independent products interferes with the amplification of the desired template-dependent products. As a result of this interference, amplification does not generate detectable quantities of all present transcripts, and the relative representation of mRNA species within the population is distorted. Because primer-dependent synthesis is constant, the abundance of template-independent synthesis becomes more of a problem as smaller amounts of RNA are used. Avoiding or minimizing the generation of these template-independent products provides a more faithful reproduction of the initial RNA population.

In at least some embodiments, the present invention identifies the presence of excess primer used in first-strand synthesis and carried over into the in vitro transcription reaction as a source of template-independent products. Reducing the amount of primer used in first-strand cDNA synthesis has surprisingly been shown to reduce the generation of primer-associated products during in vitro transcription. As a result, the information content of the amplified samples is enhanced significantly compared to protocols that result in abundant template-independent synthesis. The presence of excess primer in the in vitro transcription reaction has not previously been associated with the ability to faithfully amplify template.

Methods for eliminating the primer after completion of first-strand synthesis and prior to in vitro transcription generally have deleterious effects on the quality of the cDNA template. Attempts to remove the primer by chromatographic or electrophoretic methods result in significant loss of material. This loss of material introduces biases if the separation method does not retain all transcripts equally, regardless of sequence or length. Enzymatic treatments may result in nuclease degradation of the desired products. Therefore, there are significant drawbacks to using these approaches to reduce template-independent synthesis.

Because, as noted above, primer concentration during in vitro transcription has not previously been associated either with template-independent synthesis or with the ability to faithfully amplify template, an examination of the relationship between primer present in the in vitro transcription reaction and faithful amplification has not been undertaken in the prior art. In general, prior art amplification methods such as the polymerase chain reaction and linear amplification of mRNA utilize large molar excesses of primer, so that primer concentration does not become a limiting factor for synthesis of the desired product. Thus, the standard protocols mentioned herein call for 500 ng or more of primer regardless of the amount of starting material.

To reduce template-independent synthesis during the in vitro transcription reaction, the amount of primer used to direct first-strand cDNA synthesis by reverse transcriptase is limited. Thus, the amount of unincorporated primer carried over into the in vitro transcription reaction is also limited. In at least some embodiments, the concentration of primer in first-strand cDNA synthesis is no greater than 1 μM. In at least some embodiments, 100 ng of primer is used in a 10 μL reaction. In at least some other embodiments, 10 ng of primer is used in a 1 μL reaction. As stated above, reducing the amount of primer used in first-strand synthesis also reduces the amount of primer carried over into the in vitro transcription reaction. In at least some embodiments, no more than 0.2 μM, or no more than 0.02 μM primer is present during the in vitro transcription step.

The amount of primer carried over into the in vitro transcription reaction will vary, depending not only on the amount of primer used in the reaction, but also on the amount of available RNA template. When large amounts of template are used, more primer can be incorporated into extended products. Thus, the amount of primer carried over into the in vitro transcription reaction increases when small amounts of template are used unless the amount of primer is also reduced. The amount of template can therefore be considered in determining an appropriate amount of primer to use in first-strand synthesis. For example, 100 ng total RNA can be amplified using 100 ng or less of primer, while 2 ng total RNA can be amplified with 20 ng or less of primer. In contrast, standard amplification protocols such as those disclosed in Wang et al. (Nature Biotechnology (2000) 18: 457-459) and Luo et al. (Nature Medicine (1999) 5: 117-122) recommend 0.5-1.0 μg of primer for each first-strand synthesis reaction, even though 10 ng or less of RNA were used as starting material.

The inventors have shown that the amount of template-independent synthesis decreases significantly when the amount of primer used in the first-strand synthesis reaction is reduced in proportion to the amount of starting material used in the amplification reaction. As a result, the amount of template-independent synthesis also decreases to levels that do not interfere with template-dependent synthesis. In one non-limiting example, amplification from 2 ng of total RNA using 10 ng of primer limits the concentration of primer in the in vitro transcription reaction to no more than 0.02 μM, and synthesis of template-independent products in this reaction is limited to no more than 70 ng of total product, an amount which does not interfere with template-independent synthesis. In another non-limiting example, amplification from 200 ng of total RNA using 100 ng of primer limits the concentration of primer in the in vitro transcription reaction to no more than 0.2 μM, and synthesis of template-independent products in this reaction is limited to no more than 700 ng of total product, an amount which does not interfere with template-independent synthesis. In addition, reduction of the template-dependent product does not occur at the expense of the amplification of the template-dependent product. Instead, the optimized protocol, which uses less primer than the standard protocol, results in a greater amount of template-dependent synthesis. Thus, by significantly decreasing the amount of primer used to direct first-strand synthesis, not only is template-independent synthesis reduced; the amplification of desired mRNA species is enhanced.

To facilitate first-strand synthesis in the presence of reduced amounts of primer, at least some embodiments of the invention also decrease the reaction volume in which first-strand synthesis is performed. The first-strand synthesis reaction may be performed in the minimum volume that can reliably be measured by the instruments is less than 10 μL, often less than 5 μL, and more often less than 2 μL. The amount of starting material may be considered in determining an appropriate reaction volume, as larger starting samples of total RNA may be more faithfully amplified in larger volumes. Thus, in one non-limiting example, 100 ng of total RNA are amplified in 10 μL, while in another non-limiting example, 10 ng of total RNA are amplified in 2 μL. The reaction volume may be further reduced to any minimum volume in which the added reagents can be measured accurately by existing instrumentation. In some embodiments of the invention, the amount of primer may be reduced by limiting both the concentration of the primer and the volume of primer solution used in a first-strand cDNA synthesis reaction. By using minimal amounts of primer and controlling the volume of the reaction, the inventors have shown that production of the template independent products is correspondingly reduced, and the relative representation of mRNA species within the population is preserved.

While not being bound by any theory or mode of operation, reducing the amount of primer and/or the reaction volume may have a number of effects on reducing template-independent impurities. First, the primer carried over from the first-strand cDNA synthesis to the in vitro transcription reaction may serve as a substrate for non-templated addition of nucleotides by the enzyme to the primer, creating new sequences that are readily amplified by polymerase. Second, excess primer carried over from the first-strand synthesis reaction may itself be copied into hairpin structures similar to those observed by Biebricher and Luce (EMBO J. 15: 3458-3485, 1996), which are then efficiently amplified by the T7 polymerase, thus competing with the desired mRNA templates for enzyme and NTPs and reducing the efficiency of mRNA amplification.

The invention also provides methods for preserving the information content of a complex population of mRNA molecules by enhancing the processivity of the reverse transcriptase used for first-strand synthesis. To maximize the processivity of the reverse transcriptase, a single-strand binding protein is added at a concentration sufficient to complete synthesis of long mRNA transcripts, defined as those greater than 600 base pairs in length. Enhancing the processivity of the polymerase can be useful for amplification of any complex sample. Thus, a single-strand binding protein may be added to amplify mRNA from any amount of starting material, and using a range of primer concentrations. The single-strand binding protein may be added to reactions where microgram amounts of total RNA template are available, and where a single round of amplification is performed, but can also be added to reactions where nanogram or sub-nanogram amounts of total RNA template are used and where two or more rounds of amplification are performed.

Single-strand binding proteins may be of either prokaryotic or eukaryotic origin. Single-strand binding proteins interact with nucleic acids with little or no sequence specificity, and exhibit a preference for single-stranded nucleic acids over double-stranded nucleic acids. Proteins belonging to this group may exhibit other properties, notably an ability to disrupt or “unwind” secondary structure in single-stranded nucleic acids. Members of this group are also known to interact with and stimulate the DNA polymerases that carry out replication of dividing cells (Baker and Kornberg, DNA Replication (1992), W. H. Freeman and Company). Non-limiting examples of single-strand binding proteins are T4 gp32, E. coli SSB, retroviral nucleocapsid protein, and the eukaryotic RPA complex.

The single-strand binding protein is added to the first-strand synthesis reaction and enhances the processivity of the reverse transcriptase, thus promoting the synthesis of full-length cDNA by the reverse transcriptase. By enhancing the synthesis of all transcripts in the sample, the addition of the single-strand binding protein further contributes to the retention of information during the amplification protocol. While other groups have shown that single-strand binding proteins can stimulate the apparent overall activity of reverse transcriptase in systems that require the synthesis of short transcripts (see, e.g. Nycz et al. (1998) Analytical Biochemistry 259: 226-234; Chandler et al. (1998) Applied and Environmental Microbiology 64: 669-677), these same studies have not examined the relationship between the addition of the single-strand binding protein to reverse transcription reactions and completed synthesis of long or highly structured templates. In the methods of the present invention, templates as long as 7.5 kb in length, or 12.5-fold longer than previously demonstrated, are synthesized to completion when a single-strand binding protein is present at high concentration.

The concentration of the binding protein will vary depending on the particular protein to be used. As described below herein, the concentration of a single-strand binding protein which enhances processivity of reverse transcriptase can be determined by carrying out reverse transcription of mRNA molecules of known length and examining the products of reverse transcription using agarose gel electrophoresis and staining of the nucleic acid products. A non-limiting example of a binding protein at high concentration is T4 gp32 at a concentration of at least 0.0061 mM. The binding protein may also be used at higher concentrations, such as 0.015 mM.

In some embodiments of the present invention, the addition of single-strand binding proteins may be used in combination with strategies for priming first-strand synthesis which, in the absence of the single-strand binding protein, are associated with the loss of information encoded in the original mRNA transcript. For example, if a primer hybridizes to the 3′ end of an RNA molecule and the polymerase fails to copy the entire length of the transcript, information encoded at the 5′ end of the transcript may be lost. By improving the processivity of the polymerase, more of the sequence information from each individual transcript is maintained. Thus, the information encoded at the 5′ ends of all the species in the population, or the 5′ complexity, is better preserved when the single-strand binding protein is present. In some embodiments of the present invention, alternative priming strategies may be employed that result in the loss of information encoded at the 3′ end of the original transcript. When such priming strategies are used, the addition of the single-strand binding protein preserves the 3′ complexity of the population.

Amplification strategies using a single round of cDNA synthesis and in vitro transcription may be insufficient to yield desired amounts of material when very little starting material is available. In at least some embodiments of the present invention, a method for generating aRNA from sub-microgram amounts of starting material is provided. In the method of the invention, as little as 2 ng of total RNA may be used as starting material, and 2-8 μg of aRNA may be obtained. Assuming 3.3% poly(A)+RNA in a sample of total RNA, this represents a 120,000-fold increase in mRNA.

To generate aRNA from small quantities of total mRNA, two rounds of amplification are carried out. The first round of amplification is carried out as described above, using a limited amount of primer, a minimal reaction volume, and a single-strand binding protein at high concentration. In the second round of amplification, random priming is used to direct first-strand synthesis of cDNA from the first-round aRNA, and a single-strand binding protein is included at high concentration.

The addition of single-strand binding proteins to first-strand synthesis is useful not only for generating aRNA in the methods of the invention as described above, but for any other method that employs a reverse transcriptase to generate cDNA. Increasing the processivity of reverse transcriptase is useful for any application where reverse transcription of long or highly structured substrates is desirable. Thus, the use of a single-strand binding protein in first-strand synthesis may be employed for other applications including, but not limited to, RT-PCR, construction of cDNA libraries, and primer extension analyses.

For example, enhanced processivity of reverse transcriptase can be used to improve the 5′ complexity of cDNA libraries by promoting the completed synthesis of cDNA to be cloned into library vectors. Methods known in the art for creating cDNA libraries often contain clones lacking the sequence from the 5′ end of the original transcript due to inefficient copying by reverse transcriptase. Sequences encoded by long transcripts in particular may be very difficult to capture during library creation (see, e.g. Ausubel et al., Current Protocols in Molecular Biology, John Wiley & Sons, New York, N.Y., 1999). Enhancing the processivity of reverse transcriptase can also be useful in primer extension assays designed to map the 5′ ends of transcripts. By extending the length of transcript a reverse transcriptase can reliably copy, primer extension assays can be more readily applied to long transcripts or those that are difficult to amplify due to secondary structure. While methods such as the creation of cDNA libraries and primer extension are well known in the art, the addition of a single-strand binding protein can significantly improve the results obtained when using these methods.

In the methods of this aspect of the invention, the single-strand binding protein is supplied in any reaction where an RNA molecule is copied into DNA by reverse transcriptase at a concentration sufficient to carry out completed synthesis of templates greater than 600 nucleotides in length. Such methods may differ from the protocols for generating aRNA as described above with regard to template, primer, and further treatment of the synthesized cDNA.

It has been shown that by reducing template-independent synthesis, the methods of the invention generate aRNA that retains the information content of the starting sample. The information content of aRNA samples generated by the methods of the invention was analyzed using transcript profiling, which permits a thorough and quantitative examination of the information content contained in a complex sample of RNA molecules. It should be noted that previously published descriptions of linear amplification protocols do not provide a similarly comprehensive examination of the information content of input versus output, of the relationship between the amount of primer present in the in vitro transcription reaction, or of independently amplified samples. Furthermore, the influence of template-independent synthesis on the resulting information content has also not been examined previously.

To examine the influence of primer on template-independent synthesis, amplification products from reactions generated according previously published methods (see, e.g. Wang et al. (supra.) or Luo et al. (supra.)), referred to henceforth as the “standard protocol,” were compared to the methods of the invention, referred to henceforth as the “optimized protocol.”

Reactions according to the standard protocol were carried out in a reaction volume of 20 μL using 10 ng of template (total RNA extracted from a mixed-stage population of C. elegans) and 500 ng of primer. The in vitro transcription reaction for the standard reactions was carried out in a reaction volume of 60 μL. Reactions according to the optimized protocol were prepared in a reaction volume of 1 μL with 10 ng of template and 10 ng of primer. The in vitro transcription reaction was carried out in a reaction volume of 60 μL. Control reactions were conducted which did not include RNA template, but which did include either 500 ng or 10 ng of primer.

The amplified products were separated by agarose gel electrophoresis and stained with SYBR gold dye. Examination of the gel revealed high molecular weight products in the control reaction that included 500 ng of primer but no RNA template (see FIG. 1, lane 3). In contrast, the control reactions comprising 10 ng primer and no template did not generate these same template-independent, high molecular weight products (see FIG. 1, lane 6). Furthermore, more abundant template-dependent products were generated in the reaction using 10 ng of primer than in the reaction using 500 ng of primer (compare lanes 5 and 7 of FIG. 1). Thus, reducing the amount of primer used in standard protocols also reduced template-independent synthesis, while permitting template-dependent synthesis.

The information content of samples amplified according to the standard protocol was compared to the information content of samples amplified according to the optimized protocol. The amplified products were tested by hybridizing to the C. elegans Affymetrix GeneChip (Affymetrix, Santa Clara Calif.) and analyzing the hybridization signal. A 60% greater mean average difference, an indication of the amount of specific hybridization signal detectable over background, was detected with aRNA generated from 10 ng of total RNA with the optimized protocol, which used 10 ng of primer, than with aRNA generated from 10 ng of total RNA where 100 ng of primer was included in the first-strand synthesis reaction. The increase in mean average different indicated an overall increase in the sensitivity in transcript profiling assays provided by optimizing the amplification protocol.

To determine how many transcripts were detectably amplified with each procedure, the number of present calls from each amplification was determined from the hybridization experiment described above. Each present call is an indication that the transcript for a gene is in the mRNA sample. The average number of present calls for aRNA generated with the optimized protocol was 3495, while a sample where 100 ng of primer was used for first-strand synthesis resulted in only 322 present calls on average. The higher number of present calls for the 10-ng sample reflects the greater total diversity of information in the sample generated by the methods of the invention. It should be noted that the increase in present calls represents an improvement in sensitivity of no less than two orders of magnitude, as the optimized protocol produced a ten fold greater number of present calls, despite starting with ten-fold less input RNA. Thus, the optimized protocol permits the detection of expression of significantly more transcripts, permitting the identification of significantly more expressed genes.

The information content of amplified samples was further examined using GeneChip hybridization. Samples were compared by calculating the correlation coefficient of the data obtained from hybridization. The highest possible correlation coefficient, an indication that the data produced by two independent experiments are indistinguishable, is 1. A comparison of replicate hybridizations of the same amplified sample produced a correlation coefficient of 0.991, an indication of the variability caused by the hybridization protocol. Replicate amplifications from 10 μg of RNA, using 500 ng of primer, and 200 ng of RNA, using 200 ng of primer, produced correlation coefficients of 0.990 and 0.992 respectively. Thus, differences in the information content of parallel amplifications were similar to differences produced by the hybridization protocol itself, demonstrating that a single round of amplification produces very consistent results from one amplification to another.

These data are graphically represented in the scatter plots shown in FIG. 2. Relative frequencies of genes within the population expressed in units of parts per million (ppm) are plotted along the X- and Y-axes, with each axis corresponding to an independently hybridized and scanned sample. Each dot represents a gene and its relative frequency in each sample. Two samples with identical gene frequencies will produce a series of dots that sit exactly on the diagonal. Two samples with very close gene frequencies, as seen in FIG. 2A, will produce clusters of dots along the diagonal with some outliers. The further a dot is from the diagonal, the greater the difference in frequency of a gene in the two compared populations. FIG. 2A is a graphic representation of the comparison of the gene frequencies detected in two independent hybridizations of the same sample. Similar data were obtained from independent amplifications from 10 μg and 200 ng (compare FIG. 2A with FIGS. 2B and 2C).

The products of two-round amplification reactions were also examined in GeneChip hybridizations to determine whether independent two-round amplifications generated samples with comparable information content. Replicate double amplifications from 10 ng total RNA and 2 ng total RNA produced correlation coefficients of 0.986 and 0.984 respectively. Thus, two samples independently amplified from the same template with two rounds of amplification were slightly less alike than those amplified with one round, but still were very similar. These data are graphically represented in FIG. 2D and 2E.

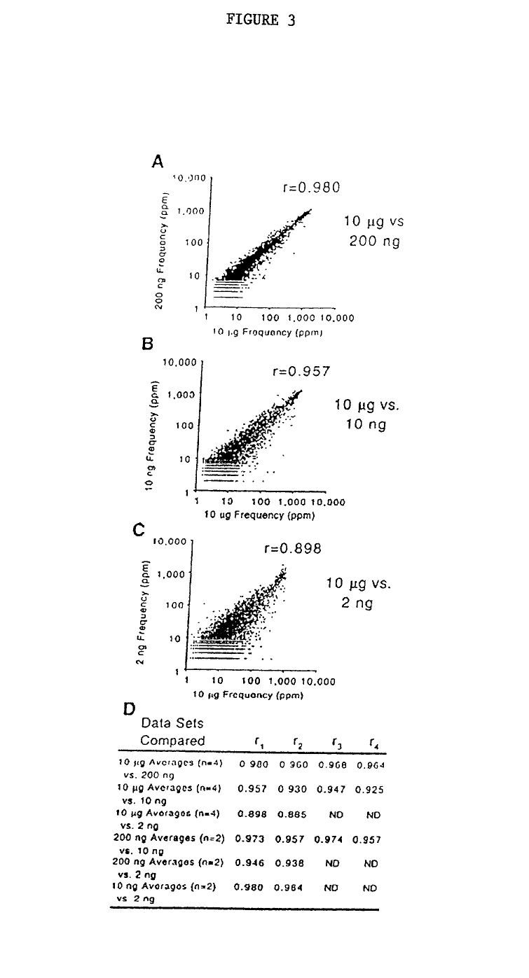

To further measure biases introduced by amplification, the correlation coefficient was calculated between profiles of a single sample of total RNA template serially diluted and amplified to microgram quantities from each dilution. The correlation coefficient between samples amplified from 10 μg and 200 ng of total RNA was 0.968; between 10 μg and 10 ng, 0.940, and between 10 μg and 2 ng, 0.892. These data are graphically represented with scatter plots in FIG. 3A, B, and C respectively.

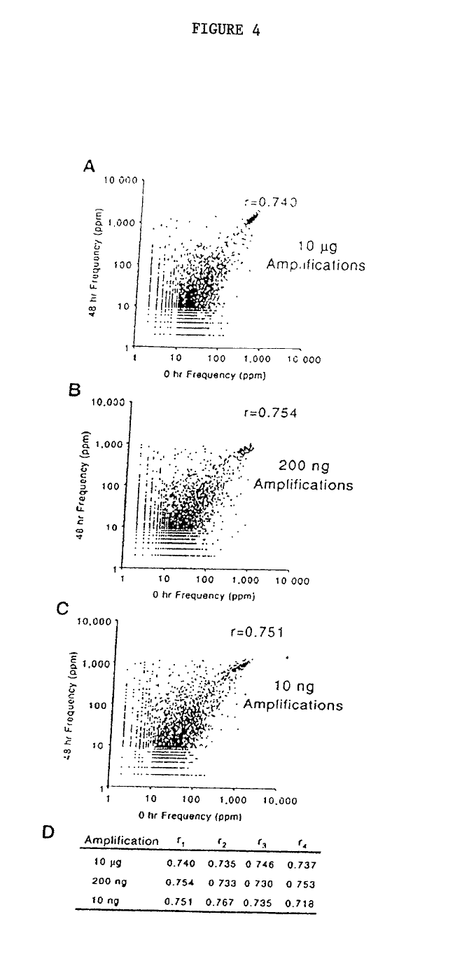

To determine whether the observed biases were sequence-dependent or copy-number dependent, the profiles of two samples generated from mRNA expressed by C. elegans animals at two different stages of development were compared and the dissimilarity between them was quantified. It was found that the two samples were as dissimilar after being amplified from 200 ng total RNA (0.743) as when amplified from 10 μg total RNA (0.734). The two samples maintain the same degree of dissimilarity even after amplification from 10 ng total RNA. In addition, the range and distribution of frequencies measured after amplification from 200, 10, and 2 ng total RNA are indistinguishable from those measured after amplification from 10 μg total RNA. The conservation of dissimilarity between RNA samples and the similarity of frequency distributions within samples suggest that amplification does not introduce copy number dependent biases. These data are graphically represented in the scatter plots shown in FIG. 4. The preservation of features that distinguish different cell types from each other indicates that conclusions drawn from experiments using transcript profiling technology will accurately reflect differences in mRNA expression.

The above experiments demonstrate that by optimizing reaction conditions for first-strand cDNA synthesis and minimizing the generation of template-independent impurities, the methods of the invention can be used to amplify complex populations of mRNA with a high degree of confidence that the amplified sample recapitulates the information content of the input sample. The methods of the invention produce reproducible data on a genome-wide scale. Such genome-wide reproducibility has not been demonstrated for other methods of amplification.

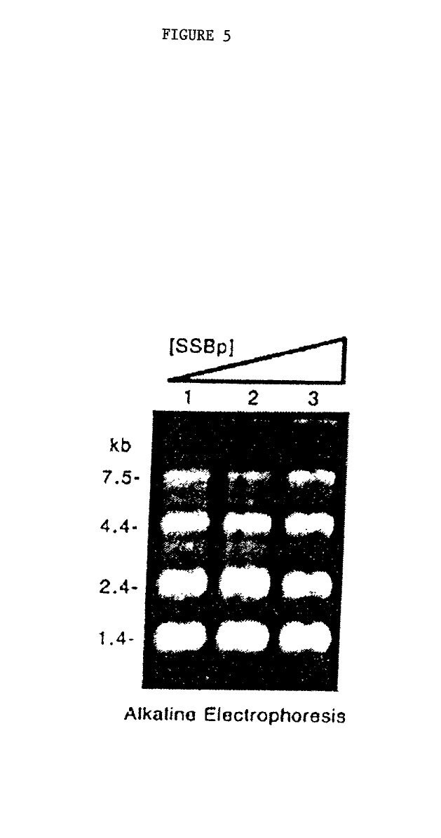

The Gene Chip used in the aforementioned experiments is designed to take into consideration the loss of information encoded by the 5′ ends of transcripts that often accompanies the amplification of mRNA populations. However, for some applications it is preferable or necessary to retain this information. Therefore, the processivity of the reverse transcriptase was analyzed and optimized in reactions that incorporated a single-strand binding protein. RNA molecular weight markers were copied into DNA with reverse transcriptase with increasing amounts of a single-strand binding protein present in the reaction. Synthesis was primed with the (dT) T7 primer used in the amplification protocols described above. 500 ng of template was used for each cDNA synthesis reaction. As shown in FIG. 5, at the highest concentration of single-strand binding protein, incomplete synthesis as measured by the loss of an “interband smear” is reduced, even for the synthesis of 7.5 and 9.5 kb RNAs (compare lane 3 to lanes 1 and 2). Thus, the information encoded in the entire sequences of these long RNA sequences is preserved. The above strategy can be used to assess the concentration of any single-strand binding protein that is sufficient to direct completed synthesis of cDNA molecules from long RNA templates.

The following examples illustrate the preferred modes of making and practicing the present invention, but are not meant to limit the scope of the invention since alternative methods may be used to obtain similar results. The issued U.S. patents, published and allowed applications, and references cited herein are hereby incorporated by reference.

### EXAMPLES

1. Isolation of Total RNA

Embryos were collected into aqueous buffer. TRIzol reagent (Life Technologies) was added and the samples were mixed vigorously to homogenize. Linear polyacrylamide was added to the reaction (GenElute LPA from Sigma) and the sample was vortexed. The sample was chloroform extracted and spun at 14K×g. The aqueous phase was transferred to a fresh tube, to which 0.7-0.8 volumes of isopropanol were added. The sample was mixed well by shaking and vortexing, and precipitated overnight at −20° C.

The precipitated sample was spun at full speed for 30 minutes at 4° C. The supernatant was removed and washed with 70% ethanol. The sample was spun again at full speed and the supernatant was removed. The pellet was dried and re-dissolved in RNase free water.

2. Synthesis of aRNA (First Round)

The sequence of the primer used for first strand synthesis was as shown in SEQ ID NO.: 1. The primer was combined with the total RNA and dried in a vacuum concentrator (Speed Vac, Savant) without drying completely. The RNA and primer were denatured at 70° C. for 4 minutes and snap cooled on ice.

Reaction components for reverse transcription were combined and added to the annealed template and primer. Final concentrations of reaction components for first-strand synthesis were as follows: 1× first strand buffer (Life Technologies), 5 mM DTT, 0.5 mM dNTPs, 200 μg/mL T4 gp32 (United States Biochemical), 1 unit RNase inhibitor, 10 units SuperScript II reverse transcriptase (Life Technologies). The sample was incubated at 42° C. for one hour. After incubating, the sample was heat inactivated at 65° C. The sample was chilled before proceeding to second strand synthesis.

Reaction components for second-strand synthesis were as follows: 1× secondstrand buffer (Life Technologies), 2 mM dNTPs, 4 units DNA polymerase I, 1 unit E. coli RNase H, 5 units E. coli DNA ligase. The reaction components for second-strand synthesis were added to the reaction containing the products of first-strand synthesis and incubated at 15° C. for two hours. Ends were polished with T4 DNA polymerase after second-strand synthesis. The reaction was heat inactivated before proceeding.

The reaction products were extracted with phenol:chloroform and added to pre-spun 0.5 mL PLG (Phase-Lock Gel, Eppendorf) tubes and spun at 13 Krpm. BioGel P-6 MicroSpin columns (Bio-Rad) were prepared according to the manufacturer's instructions. The aqueous phases from PLG were transferred to the Biogel columns and spun at 1000 g for 4 minutes. The flow through was recovered in a clean tube.

Carrier, salt, and ethanol were added for precipitation and the reaction was mixed and precipitated at −20° C. overnight. The precipitated sample was spun to pellet the RNA. The pellet was washed in 70% ethanol and air dried briefly.

Reaction components for in vitro transcription were combined. The components in the reaction were as follows: 1× Ampliscribe buffer (Epicentre Ampliscribe Kit), 7.5 mM NTP, 10 mM DTT, 30 units RNase inhibitor, 80 units HC T7 RNA polymerase (Promega).

Reaction components for in vitro transcription were added to the dried pellet and mixed to resuspend the pellet. The reaction was incubated at 42° C. for 9 hours. After incubation, the mRNA was purified by Microcon-100 (Millipore).

3. Synthesis of aRNA (Second Round).

Antisense RNA generated from first-round amplification was resuspended in water. 0.5 μg of random primers (Life Technologies) was added to the round 1 antisense RNA. The sample was dried by speed vac to a final volume of 5 μL. The sample denatured by heating and the primers were annealed by snap cooling on ice.

Reaction components for reverse transcription were prepared as for round 1. The total volume of the round 2 amplification reaction was 10 μL. The first-strand synthesis reaction was incubated as follows: 20 minutes at 37° C., 20 minutes at 42° C., 10 minutes at 50° C., and 10 minutes at 55° C. RNase H was added and the reaction was mixed, then incubated at 37° C. for 30′. The reaction was chilled on ice prior to proceeding.

Reaction components and incubation times for second-strand synthesis were the same as for first-strand synthesis.

Reaction components for in vitro transcription were as follows: 1× Ampliscribe buffer (Epicentre Ampliscribe Kit), 3.0 mM each GTP, 1.5 mM ATP, 1.2 mM UTP, 1.2 mM CTP, 0.4 mM bio-11 UTP, 0.4 mM bio-11 CTP (Enzo Laboratories), 10 mM DTT, 60 units RNase inhibitor, 160 units HC T7 RNA polymerase (Promega). The reaction was incubated at 42° C. for 9 hours. After incubation, the mRNA was purified by Microcon-100 (Millipore).

4. Measurement of RNA Concentration

aRNA was quantified either by absorbance at 260 nm or by fluorescence using Ribogreen dye (Molecular Probes) and a quartz cuvette (Hellma). Typical mass conversions for the standard protocol starting with the 10 μg total RNA and yielding RNA were 2-4 fold, for the optimized protocol starting with 50-500 ng were 10-20 fold and with 2-10 ng were 5-10 fold for the first round and 200-400 fold for the second round for a total of 30,000-120,000 fold amplification total with two rounds [assuming 3.3% poly(A)+RNA].

5. Agarose Gel Electrophoresis of aRNA.

Amplified products were separated on agarose gels. For some experiments the gels were run under native conditions; for others formaldehyde was added to denature the RNA to be analyzed. RNA was visualized by staining with SYBR gold according to the manufacturer's instructions. 6. GeneChip Hybridizations and Analysis of Hybridization Data.

1-2 μg of labeled aRNA was used in each hybridization. Hybridization and staining were performed as described in the Affymetrix Expression Analysis Technical Manual. Array images were reduced to intensity values, average differences and present/absent/marginal calls using the Affymetrix GeneChip software. Average differences were converted to relative frequencies (reported as whole numbers) by a linear fit to a standard curve, derived for each hybridization from the average difference values of eleven bacterial in vitro transcripts spiked into each hybridization at known relative frequencies ranging from 3-1000 parts per million.

Sensitivity of detection for each hybridization was defined as the relative frequency at which there was a 70% likelihood of a transcript being called present, based on a logistic regression of the present/absent calls for the spike-in transcripts as a function of the frequency. Sensitivity was 3-4 parts per million, depending on the hybridization, and there were between 3000 and 4000 present calls for each hybridization, out of 6617 predicted C. elegans genes represented on the chip.

What is claimed is:

1. A method for faithfully amplifying a complex population of mRNA molecules, said method comprising:
(a) generating double-stranded cDNA molecules from a population of RNA molecules, wherein a first-strand synthesis reaction for generating said cDNA molecules comprises a primer complementary to the mRNA molecules; and
(b) in vitro transcribing the cDNA molecules of step (a) to generate amplified antisense RNA molecules,
wherein prior to or during said in vitro transcription, the synthesis of template-independent product is reduced.

2. A method for amplifying a complex population of mRNA molecules, said method comprising:
(a) generating cDNA from a population of template RNA molecules, wherein a first-strand synthesis reaction comprises a primer complementary to the mRNA molecules; and
(b) in vitro transcribing the cDNA molecules of step (a) to generate amplified RNA molecules, wherein the primer of step (a) is present at a concentration of no greater than 0.2 μM in step (b).

3. The method of claim 2, wherein the primer from step (a) is present at a concentration of no greater than 0.02 μM in step (b).

4. The method of claim 2, wherein the primer is present at a concentration of no greater than 1 μM in step (a).

5. The method of claim 2, wherein the mRNA molecules are amplified from no more than 10 μg of total RNA.

6. The method of claim 2, wherein the mRNA molecules are amplified from no more than 1 μg of total RNA.

7. The method of claim 2, wherein the mRNA molecules are amplified from no more than 100 ng of total RNA.

8. The method of claim 2, wherein the mRNA molecules are amplified from no more than 10 ng of total RNA.

9. The method of claim 2, wherein the mRNA molecules are amplified from no more than 2 ng of total RNA.

10. The method of claim 2, wherein the mRNA molecules are amplified from total RNA isolated from fewer than 1000 cells.

11. The method of claim 2, wherein the mRNA molecules are amplified from total RNA isolated from fewer than 100 cells.

12. The method of claim 2, wherein the mRNA molecules are amplified from total RNA isolated from fewer than 10 cells.

13. The method of claim 2, wherein the mRNA molecules are amplified from total RNA isolated from a single cell.

14. The method of claim 2, wherein the primer comprises a polythymidine sequence.

15. The method of claim 2, wherein the primer comprises a promoter sequence for an RNA polymerase.

16. The method of claim 15, wherein the promoter sequence is from bacteriophage T7.

17. The method of claim 2, further comprising the steps of:
(c) generating double-stranded cDNA molecules from the antisense molecules of step (b); and
(d) in vitro transcribing the cDNA molecules of step (c) to generate amplified antisense RNA molecules.

18. A method for amplifying a complex population of mRNA molecules, said method comprising:
(a) generating double-stranded cDNA molecules from a population of mRNA molecules, wherein a first-strand synthesis reaction for generating said cDNA molecules comprises a single-strand binding protein at a concentration sufficient to support completed synthesis of templates longer than 600 nucleotides in length by a reverse transcriptase and a primer complementary to the mRNA molecules; and
(b) in vitro transcribing the cDNA molecules of step (a) to generate amplified antisense RNA molecules,
wherein the primer from step (a) is present at a concentration of no greater than 0.2 μM in step (b).

19. The method of claim 18, wherein the primer from step (a) is present at a concentration of no greater than 0.02 μM in step (b).

20. The method of claim 18, wherein the single-strand binding protein is present at a concentration of at least 0.015 mM.

21. The method of claim 18, wherein the single-strand binding protein is present at a concentration of at least 0.0061 mM.

22. The method of claim 18, wherein the single-strand binding protein comprises T4 gp32.

23. The method of claim 18, wherein the single-strand binding protein comprises the single-strand binding protein of Escherichia coli.

24. The method of claim 18, wherein the primer is present at a concentration of no greater than 1 μM in step (a).

25. The method of claim 18, wherein the mRNA molecules are amplified from no more than 10 μg of total RNA.

26. The method of claim 18, wherein the mRNA molecules are amplified from no more than 1 μg of total RNA.

27. The method of claim 18, wherein the mRNA molecules are amplified from no more than 100 ng of total RNA.

28. The method of claim 18, wherein the mRNA molecules are amplified from no more than 10 ng of total RNA.

29. The method of claim 18, wherein the mRNA molecules are amplified from no more than 2 ng of total RNA.

30. The method of claim 18, wherein the mRNA molecules are amplified from total RNA isolated from fewer than 1000 cells.

31. The method of claim 18, wherein the mRNA molecules are amplified from total RNA isolated from fewer than 100 cells.

32. The method of claim 18, wherein the mRNA molecules are amplified from total RNA isolated from fewer than 10 cells.

33. The method of claim 18, wherein the mRNA molecules are amplified from total RNA isolated from a single cell.

34. The method of claim 18, wherein the primer comprises a polythymidine sequence.

35. The method of claim 18, wherein the primer comprises a promoter sequence for an RNA polymerase.

36. The method of claim 35, wherein the promoter sequence is from bacteriophage T7.

37. A method for amplifying a complex population of RNA molecules from less than 100 ng of total RNA, said method comprising:
(a) generating double-stranded cDNA molecules from a population of RNA molecules, wherein a first-strand synthesis reaction for generating said cDNA molecules comprises a primer complementary to the mRNA molecules and a single-strand binding protein at a concentration sufficient to support completed synthesis of RNA templates greater than 600 nucleotides in length by a reverse transcriptase; and
(b) in vitro transcribing the cDNA molecules of step (a) to generate amplified RNA molecules; and
(c) generating double-stranded cDNA molecules from the antisense RNA molecules of step (b), wherein a first-strand synthesis reaction for generating said cDNA molecules comprises a single-strand binding protein at a concentration sufficient to support completed synthesis of RNA templates grater than 600 nucleotides in length by a reverse transcriptase; and
(d) in vitro transcribing the cDNA molecules of step (c) to generate amplified RNA molecules,
wherein the primer from step (a) is present at a concentration of no greater than 0.2 μM in step (b).

38. The method of claim 37, wherein the primer from step (a) is present at a concentration of no greater than 0.02 μM in step (b).

39. The method of claim 37, wherein the single-strand binding protein is present at a concentration of at least 0.015 mM.

40. The method of claim 37, wherein the single-strand binding protein is present at a concentration of at least 0.0061 mM.

41. The method of claim 37, wherein the single-strand binding protein comprises T4 gp32.

42. The method of claim 37, wherein the single-strand binding protein comprises the single-strand binding protein of Escherichia coli.

43. The method of claim 37, wherein the primer is present at a concentration of no greater than 1 μM in step (a).

44. The method of claim 37, wherein the mRNA molecules are amplified from no more than 10 ng of total RNA.

45. The method of claim 37, wherein the mRNA molecules are amplified from no more than 2 ng of total RNA.

46. The method of claim 37, wherein the mRNA molecules are amplified from total RNA isolated from fewer than 1000 cells.

47. The method of claim 37, wherein the mRNA molecules are amplified from total RNA isolated from fewer than 100 cells.

48. The method of claim 37, wherein the mRNA molecules are amplified from total RNA isolated from fewer than 10 cells.

49. The method of claim 37, wherein the mRNA molecules are amplified from total RNA isolated from a single cell.

50. The method of claim 37, wherein the primer comprises a polythymidine sequence.

51. The method of claim 37, wherein the primer comprises a promoter sequence for an RNA polymerase.

53. The method of claim 51, wherein the promoter sequence is from bacteriophage T7.

53. A method for synthesizing cDNA molecules, said method comprising a reverse transcriptase and a single-strand binding protein at a concentration sufficient to promote completed reverse transcription of mRNA molecules greater than 600 nucleotides in length.

54. The method of claim 53, wherein the single-strand binding protein is present at a concentration of at least 0.0061 mM.

55. The method of claim 53, wherein the single-strand binding protein is present at a concentration of at least 0.015 mM.

56. The method of claim 53, wherein the single-strand binding protein comprises T4 gp32.

57. The method of claim 53, wherein the single-strand binding protein comprises the single strand binding protein of Escherichia coli.

58. The method of claim 53, wherein the cDNA synthesis reaction is carried out at a temperature of no more than 42 degrees celsius.

59. A kit comprising in one or more containers:
(a) a primer comprising a sequence complementary to mRNA molecules for first-strand cDNA synthesis; and
(b) a reverse transcriptase; and
(c) a single-strand binding protein at a concentration of at least 0.25 mM.

60. The kit of claim 59, further comprising instructions for its use.

61. The kit of claim 59, further comprising instructions for limiting the synthesis of template-independent products.

62. The kit of claim 59, wherein the single-strand binding protein comprises T4 gp32.

63. The kit of claim 59, wherein the single-strand binding protein comprises the single-strand binding protein of Escherichia coli.

64. The kit of claim 59, further comprising:
(d) instructions for increasing the processivity of the RNA polymerase; and
(e) a set of control RNA molecules of several different and known lengths, suitable for the measurement of completed synthesis of mRNA transcripts longer than 600 nucleotides.

65. The kit of claim 64, wherein the control RNA molecules include RNA molecules greater than 4000 nucleotides in length.
